(19) United States
(12) Patent Application Publication
(10) Pub. No.: US 2025/0041620 A1
(43) Pub. Date: Feb. 6, 2025

(54) WEARABLE THERAPY DEVICE

(71) Applicant: LUMAFLEX LLC, Minneapolis, MN (US)

(72) Inventors: Kim Markwat, The Hague (NL); Alain Dijkstra, Amstelveen (NL); John Graham Harper, Surat Thani (TH)

(73) Assignee: LUMAFLEX LLC, Minneapolis, MN (US)

(21) Appl. No.: 18/365,222

(22) Filed: Aug. 4, 2023

(51) Int. Cl. A61N 5/06

(52) U.S. Cl. CPC A61N 5/0613; A61N 2005/0632; A61N 2005/0645; A61N 2005/0652; A61N 2005/0659; A61N 2005/0663

(57) ABSTRACT

A wearable therapy device for relieving pain and muscle's recovery, comprising a primary sheet configured with at least one slot on each side of primary sheet, each slot configured with a strap for tightening the strap with the user's body portion, a FPCB arranged in between primary sheet and secondary sheet for transmitting power to LEDs mounted on the FPCB, to illuminate, a detachable controller attached with the rear side of the primary sheet for controlling the functions, and a polarity protection circuit to alter polarity of poles.

### TECHNICAL FIELD OF THE INVENTION

The present invention relates to a wearable therapy device which is capable of providing red light therapy and infrared therapy to a user for relieving pain and for muscle recovery. In addition, the proposed device is also capable of providing magnet therapy to the user.

### PRIOR ART

US20210252301A1 disclosed methods, devices and systems which provides light therapy to human/non-human animal subjects. The invention includes shapeable light wearable therapy devices which are formable into different shapes suitable for delivery of therapeutic light to different regions of the subject's body and will retain the desired shape without a need for the use of a strap or other shape-retaining apparatus. The invention also includes light wearable therapy devices that, in at least some modes of operation, deliver light that is not visible to the human eye and which include indicator(s) to indicate to a user and/or to the subject being treated when non-visible light is actually being emitted. Although, the above prior art ‘US′301’ disclosed methods, devices and systems which are capable of providing light therapy to the human/non-human animal subjects in order to recover the subject from a disorder. However, the above prior art lacks in providing a magnet therapy to the subject. Also, the controller used in the above prior art is not detachable which when gets damage is not replaceable. The subject is required to change the whole device.

U.S. Pat. No. 10,022,554B2 disclosed a radiant energy bandage system which includes a plurality of therapeutic lamps and a controller for operating the lamps. Batteries power the lamps and are secured to a flexible fabric layer supporting the lamps and the controller. A foam and reflective layer include a spacer foam and radiant energy reflector. A plurality of spacer windows is aligned with the lamps for communicating lamp radiation therethrough. A sheer mesh fabric layer is supposed to cover the foam and reflective layer. Although, the above prior art ‘US′554’ disclosed a bandage system which provides a light therapy to the user in order to recover the user from a certain disorder. However, the above prior lacks in providing both light therapy and magnet therapy. Also, the above prior art is not capable of providing infrared therapy to the user. Further, the above prior art also lacks in monitoring health parameters of the user. Furthermore, the above prior art also lacks in preventing the user from harmful radiations.

### BACKGROUND OF THE INVENTION

The term therapy refers to a type of medical treatment which includes pain relief, muscles recovery, psychotherapy, or therapy for various mentally health disorders. Every type of therapy facilitates the person to get rid from a particular disorder. The light therapy is one of the best methods which can provide relief to a person from pain. The light therapy is also utilized for skin repairing such as removes acne. In some form of light therapy, the person is positioned near to the light therapy device. In other forms of light therapy, the therapy device is brought to near to the person's affected area for providing light therapy. One other type of therapy includes, magnet therapy in which a magnet or pair of magnets generates magnetic field which is utilized for providing magnet therapy in order to provide relief to the person from pain.

Conventionally, there are many methods are described for providing light therapy to an individual. Some of the light therapy methods include laser light or some use single LED or multiple LEDs. Earlier, the methods for providing relief to the individual from pain is generally by using light therapy. There is a lack in the prior arts that they are only providing light therapy and not facilitating the individual with the magnet therapy. Also, the conventional devices do not facilitate with polarity change of electrodes functionality which may damage the devices due to short circuit. Some of the devices are not provided with the detachable controller and also not with rechargeable batteries, and if some provided then are not provided with both wireless and wired charging system.

Therefore, there is a need in the art for therapy-based devices that do not suffer from the aforementioned deficiencies.

### OBJECTS OF THE INVENTION

Some of the objects of the invention are as follows:

An object of the present invention is to develop a device which is capable of facilitating a user with multiple light therapies in order to provide relief to the user from pain and also aid in reliving in acne like problems.

Another object of the present invention is to develop a device which automatically change the polarity of the electrodes while operating the device in order to prevent the device from being damage.

Another object of the present invention is to develop a device which is capable of providing magnet therapy to the user to provide relief to the user from pain.

Another object of the present invention is to develop a device which is configured with elastic cutouts to prevent electronic circuit from damage.

Another object of the present invention is to develop a device which is configured with a flexible and round shaped corner PCB in order to reduce the chances of breakage of the PCB.

Another object of the present invention is to develop a device which is capable to work with master/slave configuration in order to operate multiple devices.

Another object of the present invention is to develop a device which is capable of monitoring the user's vital health parameters.

Another object of the present invention is to develop a device which enables the user to operate the device with an external communication unit.

Another object of the present invention is to develop a device which facilitates the user to proceed with multiple therapies in a steady manner.

Another object of the present invention is to develop a device which is capable of protecting the user from harmful radiations.

Another object of the present invention is to develop a device which facilitates the user with day light functionality.

Yet another object of the present invention is to develop a device which is portable and re-usable.

### SUMMARY OF THE INVENTION

According to an embodiment of the present invention, there is provided a wearable therapy device. The proposed device which is water resistant, is comprising of a primary sheet having at least one elongated slot on each side of the primary sheet, wherein the elasticity of the outer edge of each of the slot is greater than that of the primary sheet. The device further includes a secondary sheet which is made of transparent silicon layer, wherein the transparent silicon layer is protruded over the primary sheet and comes in direct contact with a user to create a skin-like sensation for optimal user comfort during a therapeutic session. Further, the device includes a flexible Printed Circuit Board (FPCB) situated between the primary sheet and the secondary sheet wherein the flexible PCB includes plurality of stimulation elements which includes a plurality of LEDs and a plurality of magnetic nodes. In an alternative embodiment of the present invention, the stimulation elements include vibrating element, ultrasonic element, Pulsed Electromagnetic Field (PEMF), and electrodes, etc. The device further includes a controller detachably attached to the rear side of the primary sheet using a securing mechanism. Further, the device also integrates a polarity protection circuit to protect the device against damage caused by incorrect connection between the controller and the flexible PCB.

In one embodiment of the invention, the primary sheet or most preferably the device is shaped as oblong.

In one embodiment of the invention, the elongated slots enable the user to connect multiple devices using at least one strap.

In one embodiment of the invention, elasticity of the outer edges is increased by incorporating, but not limited to, adding a different elastic material or filing less material at the outer edges.

In one embodiment of the invention, a gap is forged between the outer periphery of the primary sheet and outer periphery of the secondary sheet.

In one embodiment of the invention, the flexible printed circuit board includes a body sensor and stimulation elements.

In one embodiment of the invention, the lights emitted by the LEDs are red and infrared light to relieve the user from pain.

In one embodiment of the invention, the edges and corners of flexible PCB are made in round shape to prevent the breakage.

In one embodiment of the invention, the securing mechanism includes, a plurality of poles to securely hold the controller and establishing the electrical connection between the controller and the flexible PCB. The securing mechanism further includes, a cavity for restricting the movement of the controller.

In one embodiment of the invention, the poles are connected to the plurality of magnetic nodes to facilitate magnetization, enabling magnetic coupling between the controller and the plurality of poles.

In one embodiment of the invention, the device is suitable to establish wired or wireless connection with user's external communication device.

In one embodiment of the invention, the controller is configured with an upper casing and lower casing.

In one embodiment of the invention, the controller is configured with a circuit board.

In one embodiment of the invention, the circuit board is attached with a rechargeable battery and a charging port.

In one embodiment of the invention, the controller is configured with an aluminum plate to restrict the radiation.

In one embodiment of the invention, the controller is configured to operate in master/slave configuration.

In one embodiment of the invention, the batteries inside the controller are supports both wired and wireless charging.

In one embodiment of the invention, the controller includes a processor, a control unit, a memory unit, a timer, a communication module, a power button, a display and a set of sensors.

In one embodiment of the invention, the method of using of wearable therapy device performed in a best manner by following some steps which includes, placing the wearable device to a user body wherein the wearable device comprises a primary sheet having at least one elongated slot on each side of the primary sheet, wherein the elasticity of the outer edge of each of the slot is greater than that of the primary sheet. The method of using wearable therapy device further includes, a secondary sheet made of transparent silicon layer wherein the transparent silicon layer is protruded over the primary sheet and comes direct contact with a user to create a skin-like sensation for optimal user comfort during a therapeutic session. Further, the method of using wearable therapy device includes, a flexible Printed Circuit Board (FPCB) situated between the primary sheet and the secondary sheet wherein the flexible PCB includes plurality of stimulation elements. Furthermore, the method of using wearable therapy device includes, detachably attaching a controller to the rear side of the primary sheet using a securing mechanism. Further, the method of using wearable therapy device includes, ensuring a proper connection between the controller and the flexible PCB to avoid damage, wherein the device integrates a polarity protection circuit to protect against incorrect connections between the controller and the flexible PCB. Further, the method of using wearable therapy device includes, activating the wearable therapy device to deliver therapeutic stimulation to the user through the stimulation elements on the flexible PCB, thereby facilitating the therapeutic session.

### BRIEF DESCRIPTION OF DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates an exploded view of a wearable therapy device, in accordance with an embodiment of the present invention.

FIG. 1B illustrates an exploded view of a securing mechanism of the wearable therapy device.

FIG. 2 illustrates a front view of the wearable therapy device, in accordance with an embodiment of the present invention.

FIG. 3A illustrates a back view of the wearable therapy device, in accordance with an embodiment of the present invention.

FIG. 3B illustrates a back view of the wearable therapy device in a stretched state, in accordance with an embodiment of the present invention.

FIG. 4 illustrates an exploded view of a controller associated with the wearable therapy device, in accordance with an embodiment of the present invention.

FIG. 5 illustrates an enlarged view of the wearable therapy device, in accordance with an embodiment of the present invention.

FIG. 6 illustrates a flow chart of the wearable therapy device, in accordance with an embodiment of the present invention.

FIG. 7 illustrates a block diagram of the wearable therapy device, in accordance with an embodiment of the present invention.

FIG. 8 illustrates a use case scenario of the wearable therapy device, with the wearable therapy device applied on the user's body portion, in accordance with an embodiment of the present invention, and

FIG. 9 illustrates another use case scenario of the wearable therapy device, with more than one wearable therapy device on the user's body portion, in accordance with an embodiment of the present invention.

### DETAILED DESCRIPTION OF THE INVENTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

In the context of this specification, terms like “light” and “illumination”, etc. refer to electromagnetic radiation in wavelength ranges varying from the visible light wavelengths (380-750 nm) to Infrared (IR) wavelengths (750 nm-1400 nm), wherein the range is inclusive of visible light and IR wavelengths. The IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, “Light Emitting Diodes (LEDs)” are envisaged to be semiconductor devices that emit electromagnetic radiation when current is applied through them. LEDs are characterized by their superior power efficiencies, smaller sizes, rapidity in switching, physical robustness, and longevity when compared with incandescent or fluorescent lamps. In that regard, the plurality of LEDs may be through-hole type LEDs (generally used to produce electromagnetic radiations of red, green, yellow, blue and white colors), Surface Mount LEDs, Bi-color LEDs, Pulse Width Modulated RGB (Red-Green-Blue) LEDs, and high-power LEDs, etc.

Materials used in the construction of LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

The proposed device is comprising of a primary sheet, a secondary sheet and a FPCB (Flexible Printed Circuit Board). The primary sheet and secondary sheet are made with silicon material. The secondary sheet is transparent in nature. The secondary sheet is made with soft material preferably made with silica gel or liquid silicon. The FPCB is wrapped in between the primary sheet and secondary sheet. Further, each of the primary sheet is crafted with an elongated slot, wherein the elasticity of each of the outer edge of the slot is higher than that of the elasticity of the primary sheet for eliminating the chance of deformation of the slot during wearing of the therapy device. Further, each of the slot is configured with a strap which is further arranged with a tightening mechanism for tightening the straps over a user's body portion. The slots are crafted in a manner that the user is allowed to connect more than one therapy device with the user's body. Further a controller, which is detachable, is attached with the rear side of the primary sheet by a securing mechanism for controlling the FPCB. The controller is configured with a plurality of female poles, whereas, the securing mechanism includes a plurality of poles and a cavity. Moreover, the poles and female poles herein also referred to electrodes. Further, a plurality of magnetic nodes are mounted on the FPCB, wherein each of the nodes are connected with each of the poles of the securing mechanism for magnetizing each of the poles. Further, each of the poles are fitted inside the female poles of the controller for holding the controller and also establishing an electrical connection between the controller and the FPCB. Furthermore, during fitting the poles within the female poles of the controller, the controller gets fitted inside the cavity of the securing mechanism which restrict the movement of the controller. Furthermore, in case, the user wrongly attached the controller with the securing mechanism, then in order to prevent the short circuit or any damage within the FPCB and controller's circuit board, a polarity protection circuit is integrated within the device to change the polarity of the poles. Further, a plurality of LEDs (Light Emitting Diodes) are mounted on the FPCB for illuminating lights of various wavelengths which includes red light and infrared light that aids in providing therapy to the user. The illumination of red light and infrared light is controlled by the user via controller or user's external communication device through wire or wireless mode. In addition, a battery which may be rechargeable is installed in the controller which provides power to the FPCB in order to illuminate the LEDs. Referring to the drawings, the present invention will now be discussed in more detail.

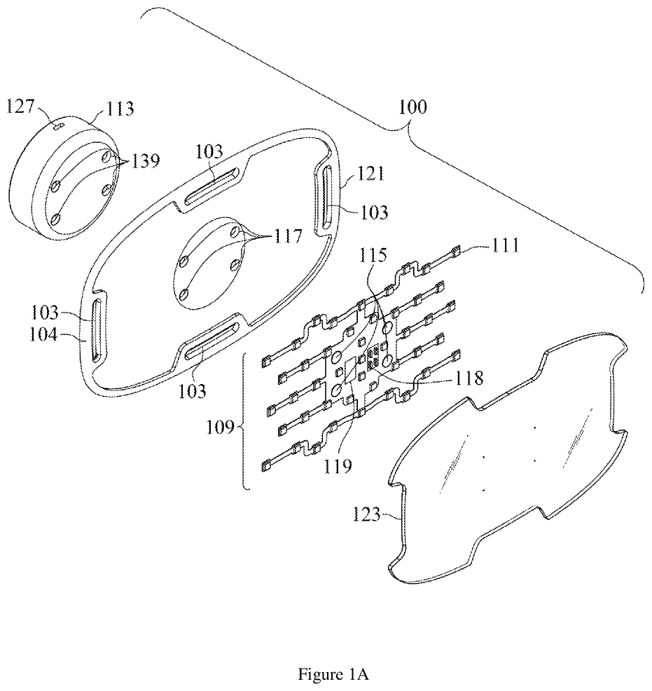

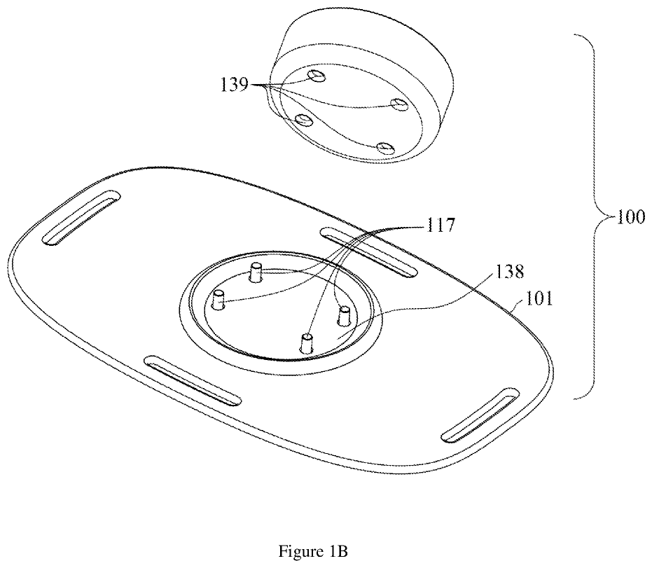

FIG. 1A illustrates an exploded view of a wearable therapy device 100 (hereinafter referred to as “the device 100”), in accordance with an embodiment of the present invention. The proposed device 100 is developed to position the oblong shaped device 100 over a particular body portion of a user. The material of the device 100 is comprising of water-resistant property which enables the device 100 to functions properly even in the wet condition. For example, in case, the user is using the device 100 during workout which includes but not limited to running, walking and skipping, the device 100 works normally and do not get affect from the sweat of the user. The water-resistant property also enables the user to use the device 100 in rainy season and also enables the user to wash the device 100. Along with water resistant properties, the material of the device 100 is also comprising of non-corrosive properties which increases the life of the device 100. In addition, the material of the device 100 is also comprising of anti-bacterial properties to prevent the user from any harmful bacteria.

Further, the proposed device 100 includes a primary sheet 121 whose peripheral edges are round, more preferably the shape of the primary sheet 121 is oblong. The primary sheet 121 covers the Flexible Printed Circuit Board (FPCB) 109 from bottom side and peripheral side of the FPCB 109 for holding the FPCB 109 securely in place.

In several embodiments of the invention, an elongated slot 103 is formed on each side of the primary sheet 121. The elasticity of outer edge 104 of each of the slots 103 is greater than that of the elasticity of the primary sheet 121. Due to high elasticity of the outer edge 104 of each slot 103, the outer edge 104 of each slot 103 is able to recover the actual shape after applying a tensile force over the slot 103 during wearing of the device 100. The high elasticity of the outer edge 104 of each of the slot 103 ensures the elimination of the chance of damage to the FPCB 109 due to force. In several embodiments of the invention, a cavity and an even number of poles 117 preferably male poles 117 are crafted at the rear side of the primary sheet 121. In several embodiments of the invention, the cavity may be rectangular, square or any other shape. In several embodiments of the invention, the poles 117 are made with ferromagnetic material or electromagnetic material.

In several embodiments of the invention, during the therapeutic session or during handling of the device 100, there are chances of damage to the FPCB 109 due to wrapping or bending. In order to reduce the chance of any damage to the FPCB 109, the corner and edges of the FPCB 109 are to be made round shape. The corners and edges in the FPCB 109 are referred to the surrounding space, where plurality of Light Emitting Diodes 111 (LEDs) are mounted.

In several embodiments of the invention, the FPCB 109 herein includes a body senor and a stimulation element which further includes the LEDs 111 and a plurality of magnetic nodes 115. Further, the FPCB 109 herein provides the secure transmission of the power to the LEDs 111. The LEDs 111 when gets power emits lights of different wavelength. Some of the LEDs 111 herein emit red light, whereas, some of the LEDs 111 emit infrared light. The LEDs 111 utilize electroluminescence phenomenon for illuminating light. The LEDs 111 are attached on the FPCB 109 at an optimum distance. In several embodiments of the invention, each of the LEDs 111 are connected through the conductive lines in which current is flowing.

In several embodiments of the invention, the LEDs 111 are utilized for providing therapy to the user's body portion in order to provide relief to the user from pain and also aids in recovering the muscle by illuminating red light and infrared light, respectively. In several embodiments of the invention, the LEDs 111 may also be utilized to help in recovering in acne, blister, and hives like disorders.

In several embodiments of the invention, the LEDs 111 emits the red light with wavelength 620 nanometer to 750 nanometers. In several embodiments of the invention, the preferred wavelength of red light is 630 nano-meters with power 15+/−5 mw/cm\hat{ }². In several embodiments of the invention, due to illumination of red light in the LEDs 111, wherein the light when comes into contact with the user's skin, the user gets relief from pain due to regeneration of cells, restoration of cells and also due to triggering of the blood flow.

In several embodiments of the invention, the LEDs 111 illuminates an infrared light with wavelength 750 nano meter to 1400 nano meters. In several embodiments of the invention, the preferred wavelength of infrared light is 850 nano-meters with power 15+/−5 mw/cm\hat{ }². Due to illumination of infrared light which when comes in contact with the user's skin and muscles, the user's muscle recovers.

In several embodiments of the invention, the plurality of magnetic nodes 115 are made with permanent magnet or may be with electromagnetic material for generating magnetic properties within the nodes 115. Due to the electromagnetic properties of the magnetic nodes 115, the nodes 115 are utilized for magnetizing the poles 117 to generate the magnetic coupling in between a controller 113 and the poles 117. In several embodiments of the invention, the magnetic nodes 115 are also utilized for providing magnetic therapy to the user via magnetic field.

Further, the body sensor 118 as described above may include but not limited to a heart rate sensor, blood pressure sensor, sleep tracker sensor, blood oxygen sensor, skin surface pH sensor, skin conductance sensor and temperature sensor. The body sensors 118 are used herein for monitoring various parameters in the user's body such as, temperature, sleep pattern and heart rate, oxygen level, skin disorders, and blood pressure etc. In several embodiments of the invention, during monitoring the user's heart beat by the heart rate sensor, the LEDs 111 blinks automatically as per the heartbeat of the user in a synchronous manner. In an example, in case, the user's heartbeat is 80 times per minute, then the LEDs 111 will blink with the frequency 1.33 Hz/s i.e. in 1 second the LEDs 111 will blink for 1.33 times. Similarly, in case, the user's heartbeat is 70 times per minute, then the LEDs 111 will blink with the frequency 1.16 Hz/s. Thereby, providing the user a visual experience of increasing/decreasing the heartbeats.

In several embodiments of the invention, the proposed device 100 also includes a secondary sheet 123 which is transparent covers the FPCB 109 from top side of the FPCB 109. In several embodiments of the invention, the surface of the secondary sheet 123 is smooth. The surface of the secondary sheet 123 provides smooth touch to the user during a therapeutic session. The secondary sheet 123 herein is made to be extra soft preferably made with silica gel or liquid silicon so that the user during the therapeutic session feels like another human's like skin.

Further, in several embodiments of the invention, the controller 113 is detachably attached to the rear side of the primary sheet 121. In several embodiments of the invention, the controller 113 herein is detachable which makes the device 100 portable and also save the cost in case of any fault in the controller 113. The user is required to detach the controller 113 from the primary sheet 121, when the controller 113 is faulty, so that the device 100 is able to function properly with the repaired controller 113 or a new controller 113. In several embodiments of the invention, an even number of a plurality of female poles 139 are configured with the controller 113 which comes in direct contact with the magnetized poles 117 to transmit the power and establishing a secured electric connection between the controller 113 and FPCB 109.

In several embodiments of the invention, the magnetized poles 117 and female poles 139 are utilized for providing strong bond in between the FPCB 109 and the controller 113. In several embodiments of the invention, the female poles 139 and magnetized poles 117 make strong grip due to the attraction force which is generated by the contact of south pole and north pole within the female poles 139 and magnetized poles 117, respectively or vice versa. For example, the south pole of the female poles 139 comes in direct contact with the north pole of the magnetized poles 117, an attractive force will be generated among the female poles 139 and magnetized poles 117. In several embodiments of the invention, the strong grip between the female poles 139 and magnetized poles 117 is further aided with the attachment of the controller 113 with the cavity for restricting the movement of the controller 113 in multi-directions. In several embodiments of the invention, the controller 113 herein is crafted with a charging port 127. In several embodiments of the invention, the controller 113 is also inbuilt with a wireless charger.

In several embodiments of the invention, in case, the user wrongly attaches the controller 113 with the poles 117, then in order to prevent the FPCB 109 and controller 113 from being damage due to short circuit, a polarity protection circuit 119 is integrated may be within both the controller 113 and the FPCB 109, to alter/changes the polarity of the poles 117 and female poles 139, thereby prevent the device 100 from being damage.

FIG. 1B illustrates an exploded view of a securing mechanism 138 of the wearable therapy device 100, in accordance with an embodiment of the present invention. The securing mechanism 138 is incorporated into the primary sheet 121 for detachably attaching the controller 113 to the rear side of the primary sheet 121. The controller 113 and a wearable pad 101 which includes primary sheet 121, FPCB 109, and secondary sheet 123, are shown. The female poles 139 inside the controller 113 is shown which are in detachable manner. Further, the poles 117 which are preferably male poles 117 on the pad 101 are shown. In order to attach the controller 113 with the pad 101, the male poles 117 get inserted into the female poles 139 which results in making a grip between the pad 101 and the controller 113, and results in establishing an electrical connection between the controller 113 and FPCB 109.

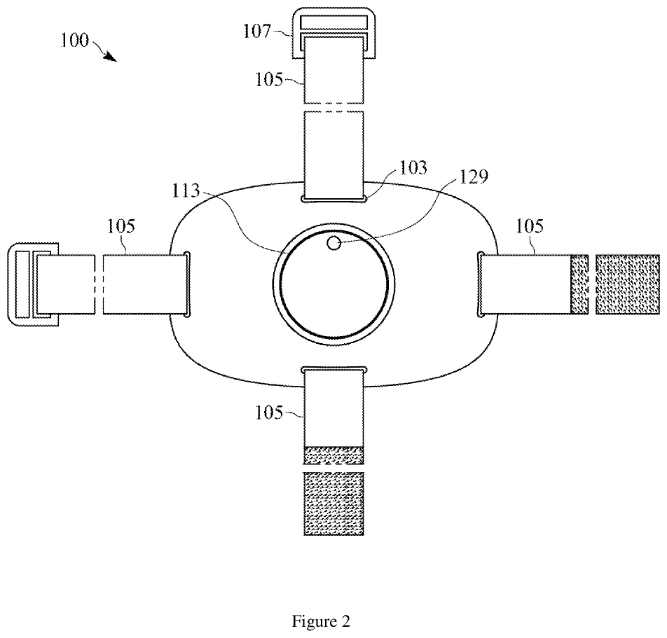

FIG. 2 illustrates a front view of the wearable therapy device 100, in accordance with an embodiment of the present invention. Each of the slot 103 in FIG. 2 is attached with a strap 105. The user is required to attach the number of straps 105 attached with the slots 103 as per the need or the requirement of the user. For example, the user is using a single therapy device 100 then the number of straps 105 used will be two. In another example, if the user is using two devices 100 then the user is required to use three straps 105 at each device 100.

Further, the straps 105 are made with a flexible and soften material. The material of the straps 105 is comprising of elastic properties which eliminates the chance of any damage to the straps 105 as due to this elastic property, the strap 105 is able to restore the length of the strap 105 after loosening of the strap 105 from the user's body portion. For example, the length of strap 105 is ‘x’ cm, the user is fastening the straps 105 around the biceps of the user, then the length of the straps 105 increases to ‘y’ cm. Now, due to elastic properties of the strap's 105 material even after unfastening of the straps 105, the length of the strap 105 remains same i.e., ‘x’ cm.

Further, each of the strap 105 is linked with a tightening mechanism 107 which is preferably a Velcro which is comprises of a hook and loop. The user when tight the strap 105 with the user's body portion attach the hook portion of the tightening mechanism 107 with the loop portion.

In several embodiments of the invention, a power button 129 is associated with the controller 113 for powering on/off the device 100. Upon switching on the button 129, the user is allowed to take the therapeutic session as per the desire of the user. For powering on the device 100, the user is required to press the button 129 for 1.5 seconds. Then, in case, the user again presses the button 129, the treatment mode for 10 minutes will be switched on. In case, the user presses the button 129 in between the treatment then the treatment will be paused. Further, after finishing 10 minutes treatment, the device 100 automatically gets switched off. In several embodiments of the invention, the device 100 will automatically be switched off when the device 100 is switched on and not in use.

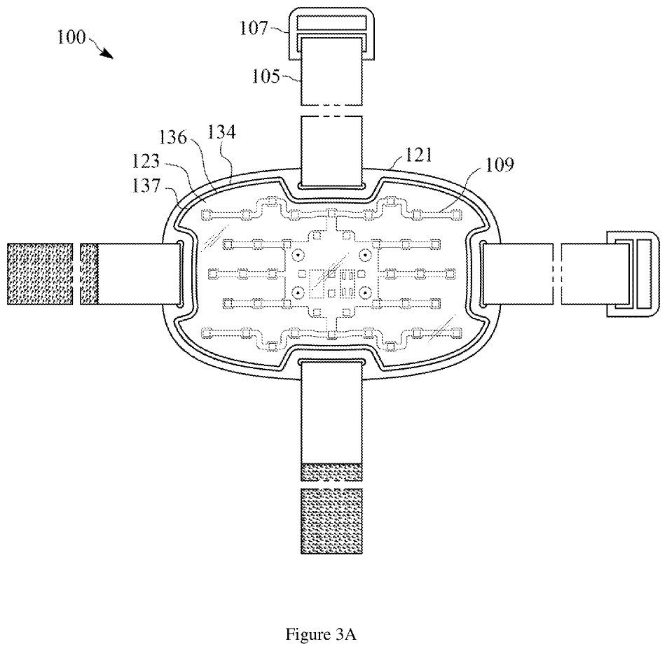

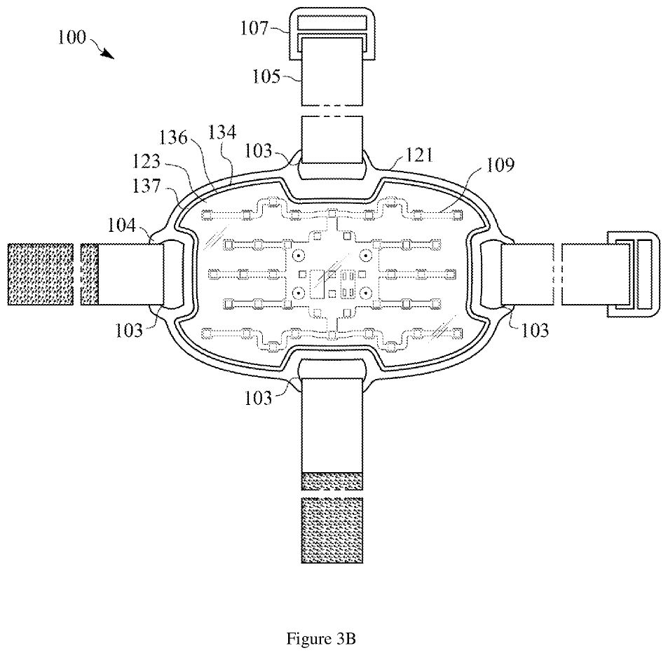

FIGS. 3A and 3B illustrates a back view of the wearable therapy device 100, a back view of the wearable therapy device 100 in a stretched state, respectively, in accordance with an embodiment of the present invention. The FIGS. 3A and 3B includes the secondary sheet 123, primary sheet 121, FPCB 109, straps 105 and tightening mechanism 107. There is a little gap 137 provided in between the outer periphery 136 of the secondary sheet 123 and outer periphery 134 of the primary sheet 121 which is utilized for separating the secondary sheet 123 from the primary sheet 121. The gap 137 herein is also utilized for allowing the secondary sheet 123 to expand. For example, during taking the therapeutic session, the secondary sheet 123 directly comes in the contact with the user's body portion. The secondary sheet 123 upon tightening of the strap 105 experience a compressive force due to which the secondary sheet 123 start expanding in outward direction. In order to allow the expansion of the secondary sheet 123, the gap 137 is provided in between the outer periphery 134 of the primary sheet 121 and outer periphery 136 of the secondary sheet 123. In several embodiments of the invention, the gap 137 may also be utilized for extracting heat which may be associated during the therapeutic session, to the environment.

In several embodiments of the invention, in FIG. 3B the outer edges 104 of the slots 103 are stretching due to the pulling of the straps 105 by applying a tensile force in the outwards direction. The elasticity of the outer edges 104 of the slots 103 is high which is made with filling of less material at the outer edges 104 or may be made with different material as compare to the material of the primary sheet 121. The FPCB 109 remains secured during the stretching of the slots 103 as due to high elasticity of the outer edges 104 of the slots 103, there is negligible effect of the stretching on the FPCB 109. Further, upon removing of the applied force, the slots 103 will come to original dimensions due to high elasticity of the outer edges 104.

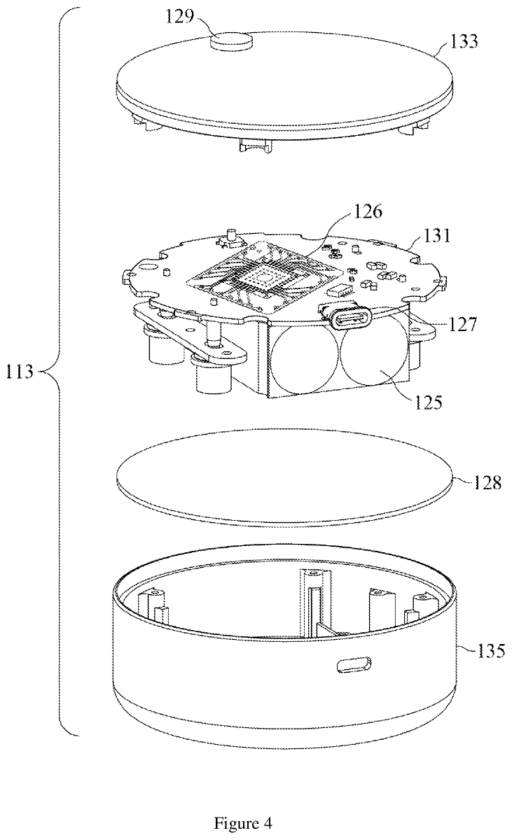

FIG. 4 illustrates an exploded view of a controller 113 associated with the wearable therapy device 100, in accordance with an embodiment of the present invention. The controller 113 herein includes an upper casing 133 which is configured with the button 129. The shape of the outer edges of the upper casing 133 is preferably circular and alternatively may be square or may be of any other shape same as the shape of cavity. The button 129 provided herein allows the user to control the device 100. In several embodiments of the invention, the controller 113 also includes a lower casing 135 which is to be attached with the primary sheet 121 by the user.

In several embodiments of the invention, inside the controller 113, a circuit board 131 is configured which is comprises of conductive lines for transmitting the power from a battery 125 which is attached with the circuit board 131, to the FPCB 109. The charging port 127 is attached with the circuit board 131 and the batteries 125 which allows the user to recharge the batteries 125. Each of the batteries 125 is preferably a lithium-ion battery 125 that provides power to the FPCB 109. In several embodiments of the invention, an aluminum plate 128 is mounted below the circuit board 131 for restricting the flow of radiations generated due to the electronic components mounted on the circuit board 131, thereby protecting the user from harmful radiations.

In several embodiments of the invention, the controller 113 is comprising of a processor 126 which includes a memory unit, a control unit and a set of sensors. The memory unit stores therapeutic programs which are pre-fed within the memory unit. Upon receiving the command from the user, the device 100 works on the basis of therapeutic program stored in the memory unit. The controller 113 herein is configured with the master/slave configuration which is utilized when the user is using multiple devices 100 at a single time. The master device 100 and slave devices 100 are connected with each other through wireless connection. For example, in case the user wears multiple devices 100 at the user's body portions. Then, among all devices 100 there will be a master device 100 which when gets input, execute the input and accordingly through wireless connection, the other attached slave devices 100 will also execute the same input or may be different as per the command of the user through the master device 100.

In several embodiments of the invention, each of the female poles 139 of the controller 113 are interlinked with magnets to aid in gripping the controller's 113 female poles 139 with the poles 117 attached with the FPCB 109. In several embodiments of the invention, at the lower side of the controller 113, an aluminum plate 128 is configured, which aids in restricting the radiations emitted from the components mounted on the controller's 113 circuit board 131, and direct the radiation towards the FPCB 109.

In several embodiments of the invention, the processor 126 includes a polarity sensor which monitors the negative, positive polarity of the female poles 139 which is changed by the polarity protection circuit 119 in case of wrong attachment of the controller 113 with the FPCB 109. In several embodiments of the invention, the controller 113 wirelessly connects with the body sensor 118 which is present within the FPCB 109 that enables the controller 113 to automatically works as per the data monitor by the body sensor 118. For example, in case, the body sensor 118 detects that the user's heart beat is very fast, then the controller 113 automatically stops the power transmission to the FPCB 109 in order to stop the therapeutic session.

In several embodiments of the present invention, the device 100 also facilitates the user with day light functionality. In an example, by analyzing the user's sleeping pattern by the body sensor 118, the controller 113 automatically turns on the light at the wake-up time of the user and also slowly increase the brightness level of the LEDs 111 accordingly to the sun rising. In another example, by analyzing the user's sleeping pattern by the body sensor 118, the controller 113 automatically turns off the light at the sleeping time of the user. In another example, in case, two or more devices 100 are attached with the user' body portions, and the body sensor 118 from one of those attached devices 100 detect the pain in a particular veins of the user, then the body sensor 118 of another device 100 automatically detect that veins and accordingly provide the therapy on that particular vein.

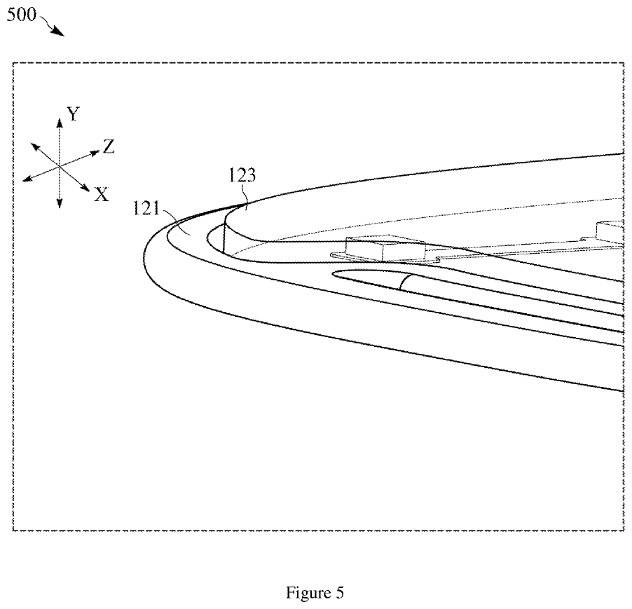

FIG. 5 illustrates an enlarged view of the wearable therapy device 100, in accordance with an embodiment of the present invention. In several embodiments of the invention, the surface of the secondary sheet 123 is outwardly protruding from the primary sheet 121 in y-direction as shown in FIG. 5. Due to protruded surface of the secondary sheet 123, the contact between the secondary sheet 123 and the user's body portion is proper which facilitate the user to take therapy comfortably.

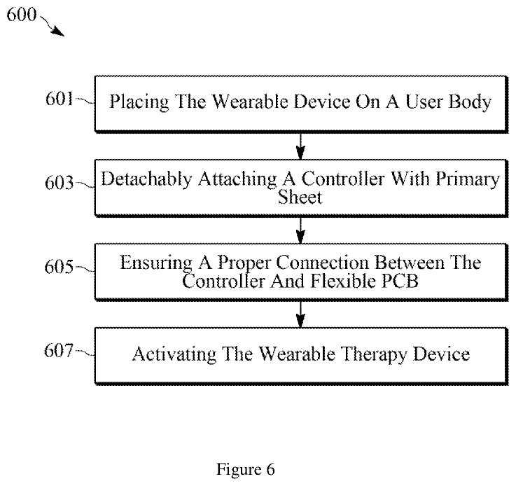

FIG. 6 illustrates a flow chart 600 of the wearable therapy device 100, in accordance with an embodiment of the present invention. The flow chart 600 describes the step by step information to guide the user to use the device 100. In the first step 601, placing the wearable device 100 on a user body which is comprising of a primary sheet 121 providing with an elongated slot 103 on each side of primary sheet 121. The wearable device 100 is further comprising of a secondary sheet 123 which is transparent. The second step 603 includes, detachably attaching a controller 113 with primary sheet 121 through the cavity and by joining poles 117 with female poles 139. The third step 605 includes, ensuring a proper connection between the controller 113 and the FPCB 109. The final step 607 includes, activating the wearable device 100 in order to enable the user to proceed with the therapeutic session.

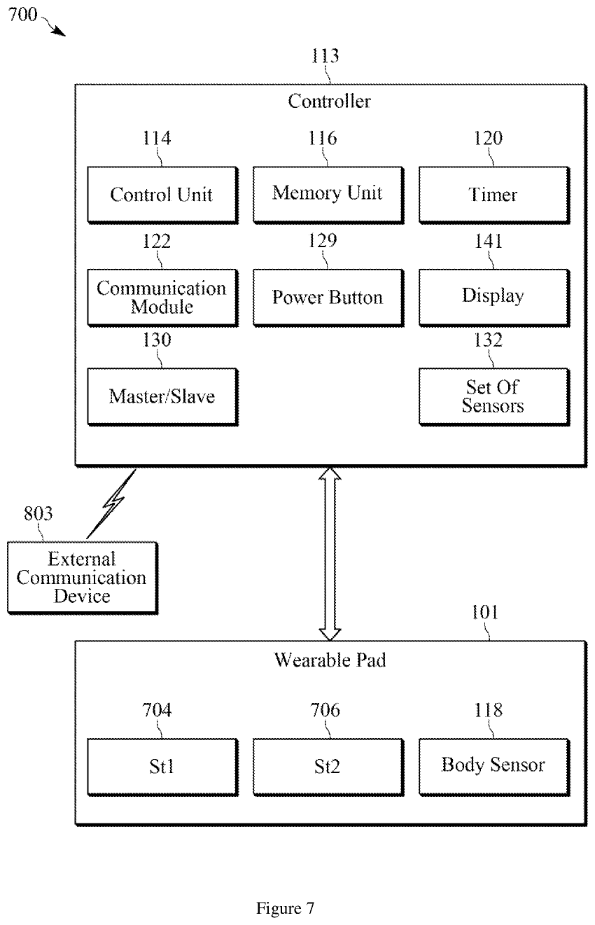

FIG. 7 illustrates a block diagram 700 of the wearable therapy device 100, in accordance with an embodiment of the present invention. The block diagram 700 shows that the controller 113 and wearable pad 101 is interlinked with each other. The controller 113 herein includes a control unit 114, a memory unit 116, a timer 120, a communication module 122, a power button 129, a display 141, a master/slave 130 configuration and a set of sensors 132. The controller 113 is allowed to control the wearable pad 101 via an external communication device 803 and by the power button 129 mounted on the controller 113. The controller 113 and external communication device 803 are connected and allowed to communicate with each other via communication module 122. The control unit 114 allows the user to control the wearable pad 101 in order to take a therapeutic session. The memory unit 116 is pre-fed with the therapeutic programs which may include but not limited to proceed with only red therapy, proceed with only infrared therapy, proceed with both red and infrared therapy, proceed with only magnet therapy, and proceed with both light and magnet therapy. Further, a timer 120 is inbuilt within the controller 113 which is interlinked with the memory unit 116. The timer 120 enables the user to choose the therapy time as per the user's convenience. The controller 113 is also provided with a display 141 on which the user is allowed to select mode of therapy, time of therapy, and any other notifications. Further, the controller 113 is also provided with master/slave 130 configuration via which a master device 100 is allowed to connect with another slave device 100 wirelessly. The set of sensors 132 may include but not limited to polarity sensor, and connected sensor. The polarity sensor is allowed to change the polarity of the female poles 139 in case of wrong connection of the controller 113 with the FPCB 109. The connected sensor makes the connection in between the controller 113 and the wearable pad 101. Further, the wearable pad 101 includes stimulation element 1 (St1) 704, stimulation element 2 (St2) 706, body sensor 118. The St1 704 and St2 706 may include red light therapy or infrared light therapy or magnet therapy. The body sensor 118 is allowed to monitor the vital health parameters of the user.

In another embodiment of the present invention, the device 100 is encrypted with artificial intelligence protocols which is synchronized with the pad 101, controller 113 and external communication device 803, wherein the artificial intelligence monitors various parameters which includes but not limited to user's daily schedule, types of therapy required to the user as per the health of the user, sleeping pattern, heart rate, and other parameters. In an example, the user daily takes a therapeutic session at a particular time, and in case, the user forgot or is delayed to take the session, then a notification will be sent to the user's external communication device 803 through artificial intelligence. In another embodiment of the present invention, the artificial intelligence also tracks the user's health parameters and accordingly send a notification on the user's external communication device 803 to notify the user about the type of therapy required to the user according to the health of the user. In another embodiment of the present invention, the artificial intelligence also facilitates the user to select the mode of therapy required according to the health of the user. In another embodiment of the present invention, the artificial intelligence automatically adjusts the intensity and duration of the therapy as required to the user as accordance to the health of the user.

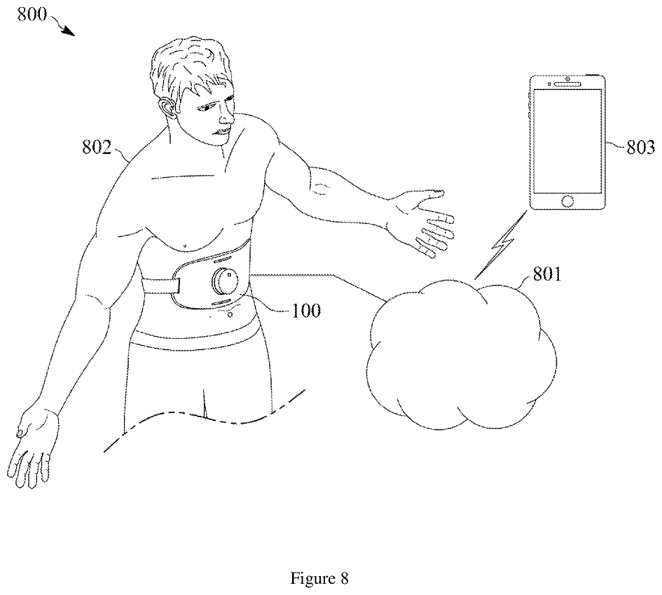

FIG. 8 illustrates a use case scenario 800 of the wearable therapy device 100, with the wearable device 100 applied on the user's body portion, in accordance with an embodiment of the present invention. In the use case scenario 800, the user 802 is shown with wearing the wearable therapy device 100 over the stomach portion of the user 802. The proposed device 100 is wirelessly connected to an external communication device 803 which includes but not limited to mobile and laptop. In FIG. 8, the external communication device 803 shown is a mobile which is connected with the device 100 with a wireless network.

In several embodiments of the invention, the signals or controls are transmitted in between the wearable therapy device 100 and the mobile via cloud computing 801 which allows the user 802 to securely choose the type of therapy, the user 802 is desire to use. For example, in case, the user's 802 desire is to take red light therapy, then the user 802 is required to select the option in the external communication device 803. Similarly, the user's 802 desire is to take infrared therapy, then select the infrared therapy option from the list of therapy shown within the external communication device 803. Similarly, the user's 802 desire is to take magnet therapy, then select the magnet therapy option from the list of therapy shown within the external communication device 803.

In several embodiments of the invention, the external communication device 803 includes another selection mode which allows the user 802 to take the therapeutic session as per the desire of the user 802. For example, in case, the user 802 desire to take red light therapy and the infrared light therapy together, then the user 802 is required to select the red-light therapy and the infrared light therapy mode from the selection mode. In several embodiments of the invention, a timer 120 is also included within the external communication device 803 which allow the user 802 to select the time for therapeutic session which when selected automatically stop the therapeutic session.

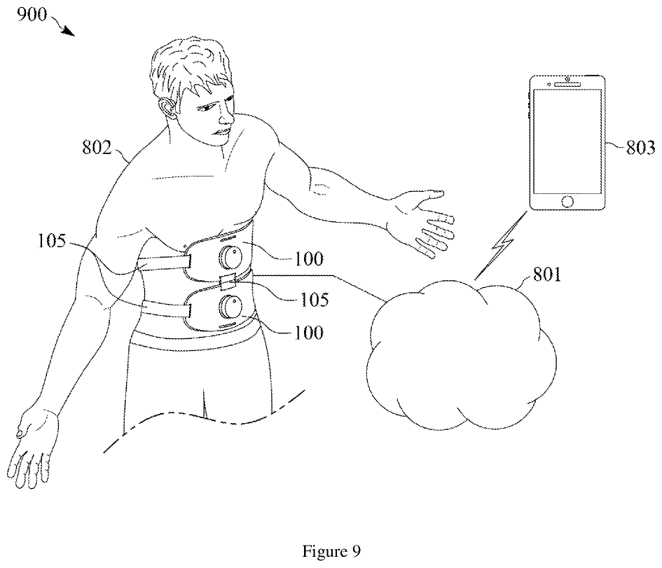

FIG. 9 illustrates another use case scenario 900 of the wearable therapy device 100, with more than one wearable therapy device 100 on the user's 802 body portion, in accordance with an embodiment of the present invention. In the use case scenario 900, the user 802 is shown with wearing a pair of wearable therapy device 100 over the stomach portion of the user 802. The proposed device 100 is wirelessly connected to the external communication device 803 which includes but not limited to mobile and laptop. In FIG. 9, the external communication device 803 shown is a mobile which is allowed to connect with the device 100 with a both wired and wireless network.

In several embodiments of the invention, the signals or controls are transmitted in between the wearable therapy device 100 and the mobile via cloud computing 801 which allows the user 802 to securely choose the type of therapy, the user 802 desire to use. For example, in case, the user's 802 desire is to take red light therapy, then the user 802 is required to select the option in the external communication device 803. Similarly, the user's 802 desire is to take infrared therapy, then select the infrared therapy option from the list of therapy shown within the external communication device 803. Similarly, the user's 802 desire is to take magnet therapy, then select the magnet therapy option from the list of therapy shown within the external communication device 803.

In several embodiments of the invention, the external communication device 803 includes another selection mode which allows the user 802 to take the therapeutic session as per the desire of the user 802. For example, in case, the user 802 desire to take red light therapy and the infrared light therapy together, then the user 802 is required to select the red-light therapy and the infrared light therapy mode from the selection mode. In several embodiments of the invention, the selection mode in the external communication device 803 enables the user 802 to operate both the devices 100 at a single time with alternatively or together taking the red-light therapy and the infrared light therapy. In several embodiments of the invention, the timer 120 in the external communication device 803 allow the user 802 to select the time for therapeutic session which when selected automatically stop the therapeutic session. In several embodiments of the invention, the user 802 is allowed to control the device 100 from any of the controller 113 and also from the external communication device 803.

The invention as described above offers several advantages. For instance, the wearable therapy device is very simple in design and construction. Further, the wearable therapy device uses commonly available materials. The simplicity in design and construction, and the use of commonly available materials allow the wearable therapy device to be mass-produced with minimal capital expenditure, and to be made available in the market at significantly lower prices. Also, the wearable therapy device can be reused several times and is, therefore, cost-effective for the end user and minimizes waste generation.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to be providing the broadest scope consistent with the principles and the novel and inventive features disclosed or suggested herein. Accordingly, the invention is anticipated to hold on to all other such alternatives, modifications, and variations that fall within the scope of the present invention and appended claims.

1. A wearable therapy device, comprising:
a primary sheet having at least one elongated slot on each side of said primary sheet, wherein the elasticity of the outer edge of each of said slot is greater than that of said primary sheet;
a secondary sheet made of transparent silicon layer wherein the transparent silicon layer is protruded over said primary sheet;
a flexible Printed Circuit Board (FPCB) situated between said primary sheet and said secondary sheet wherein the said flexible PCB includes plurality of stimulation elements;
a controller detachably attached to the rear side of said primary sheet using a securing mechanism; and,
wherein said device integrates a polarity protection circuit to protect said device against damage caused by incorrect connection between said controller and said flexible PCB.

2. The wearable therapy device as claimed in claim 1, wherein said primary sheet is shaped as oblong.

3. The wearable therapy device as claimed in claim 1, wherein said elongated slots enable the user to connect multiple said devices using at least one strap.

4. The wearable therapy device as claimed in claim 1, wherein elasticity of said outer edges is increased by incorporating, but not limited to, adding a different material or filing less material.

5. The wearable therapy device as claimed in claim 1, wherein a gap is forged between the outer periphery of the said primary sheet and outer periphery of said secondary sheet.

6. The wearable therapy device as claimed in claim 1, wherein the flexible printed circuit board includes a plurality of Light Emitting Diodes (LEDs), a body sensor and a plurality of magnetic nodes.

7. The plurality of LEDs as claimed in claim 6 are emitting red and infrared light.

8. The flexible PCB as claimed in claim 6 has edges and corners made in a round shape to prevent breakage.

9. The wearable therapy device as claimed in claim 1, wherein the securing mechanism comprises;
a plurality of poles to securely hold the controller and establishing the electrical connection between said controller and said flexible PCB; and,
a cavity to restrict movement of said controller during affixing of said controller with said primary sheet.

10. The plurality of poles as described in claim 9 are connected to the plurality of magnetic nodes to facilitate magnetization, enabling magnetic coupling between the controller and the plurality of poles.

11. The device disclosed in claim 1, wherein said device is wirelessly connected with user's external communication device which enables said user to control said device from said external communication device.

12. A method of using a wearable therapy device comprising the steps of:
a) placing said wearable device to a user body, wherein the said wearable device comprises:
a primary sheet having at least one elongated slot on each side of said primary sheet, wherein the elasticity of the outer edge of each of said slot is greater than that of said primary sheet;
a secondary sheet made of transparent silicon layer wherein the transparent silicon layer is protruded over said primary sheet;
a flexible Printed Circuit Board (FPCB) situated between said primary sheet and said secondary sheet wherein the said flexible PCB includes plurality of stimulation elements;
b) detachably attaching a controller to the rear side of said primary sheet using a securing mechanism;
c) ensuring a proper connection between said controller and said flexible PCB to avoid damage, wherein the device integrates a polarity protection circuit to protect against incorrect connections between the controller and the flexible PCB; and
d) activating the wearable therapy device to deliver therapeutic stimulation to the user through the stimulation elements on the flexible PCB, thereby facilitating the therapeutic session.

13. The method as claimed in claim 12, wherein said elongated slots enable the user to connect multiple said devices using at least one strap.

14. The method as claimed in claim 12, wherein elasticity of said outer edges is increased by incorporating, but not limited to, adding a different material or filing less material.

15. The method as claimed in claim 12, wherein the flexible printed circuit board includes a plurality of Light Emitting Diodes (LEDs), a body sensor and a plurality of magnetic nodes.

16. The plurality of LEDs as claimed in claim 15 are emitting red and infrared light.

17. The flexible PCB as claimed in claim 15 has edges and corners made in a round shape to prevent breakage.

18. The method as claimed in claim 12, wherein the securing mechanism comprises;
a plurality of poles to securely hold the controller and establishing the electrical connection between said controller and said flexible PCB; and,
a cavity to restrict movement of said controller during affixing of said controller with said primary sheet.

19. The plurality of poles as described in claim 18 are connected to the plurality of magnetic nodes to facilitate magnetization, enabling magnetic coupling between the controller and the plurality of poles.

20. The method as claimed in claim 12, wherein said device is wirelessly connected with user's external communication device which enables said user to control said device from said external communication device.
